(19) United States
(12) Patent Application Publication
(10) Pub. No.: US 2009/0324715 A1
(43) Pub. Date: Dec. 31, 2009

(54) HIGH DOSE ORAL PHARMACEUTICAL COMPOSITIONS OF ARTEMETHER AND LUMEFANTRINE

(76) Inventors: Sumit Madan, New Delhi (IN); Vikas Batra, Karnai (IN); Arno Appavoo Enose, Kanyakumari (IN); Vinod Arora, New Delhi (IN)

Correspondence Address: Ranbaxy Inc., Intellectual Property Department, 600 College Road East, PRINCETON, NJ, 08540, US

(21) Appl. No.: 12/305,087

(22) PCT Filed: Jun. 22, 2007
(86) PCT No.: PCT/IB2007/052436

§ 371 Date: Apr. 3, 2009

(30) Foreign Application Priority Data
Jun. 26, 2006 (IN) 1492/DEL/2006

(51) Int. Cl. A61K 31/335; A61P 33/06; A61K 9/44

(52) U.S. Cl. 424/467; 514/450

(57) ABSTRACT

The present invention relates to high dose oral pharmaceutical compositions of artemether and lumefantrine, and process for preparation thereof. The compositions comprise of artemether and lumefantrine comprising artemether in an amount of from about 40 mg to about 80 mg, lumefantrine in an amount of from about 240 mg to about 480 mg. The compositions are useful for treatment of uncomplicated infections with Plasmodium falciparum, including strains from multi-drug-resistant areas.

### FIELD OF THE INVENTION

The present invention relates to high dose oral pharmaceutical compositions of artemether and lumefantrine, and process for preparation thereof. The compositions comprise of artemether and lumefantrine comprising artemether in an amount of from about 40 mg to about 80 mg, lumefantrine in an amount of from about 240 mg to about 480 mg. The compositions are useful for treatment of uncomplicated infections with Plasmodium falciparum, including strains from multi-drug-resistant areas.

### BACKGROUND OF THE INVENTION

Malaria is a parasitic disease spread by mosquitoes. It affects millions of people worldwide and causes significant illness and mortality. Uncomplicated malaria presents with symptoms such as fever, headache, muscle pain, and vomiting. The parasite has become resistant to a number of previously effective drugs, and so combinations of drugs have been used to try to prevent further resistance. Artemether-lumefantrine is one such drug combination, which can be used for the treatment of uncomplicated infections with P. falciparum, including strains from multi-drug resistant areas.

Lumefantrine is a racemic fluorene derivative with the chemical name 2-dibutylamino-1-[2,7-dichloro-9-(4-chlorobenzylidene)-9H-fluoren-4-yl]-ethanol. It conforms, structurally, to the aryl-amino alcohol group of antimalarials including quinine, mefloquine and halofantrine.

Artemether is a methyl-ether derivative of dihydroartemisinin derived from artemisinin (Qinghaosu), chemically described as (3R,5aS,6R,8aS,9R,10S,12R,12aR)-Decahydro-10-methoxy-3,6,9-trimethyl-3,12-epoxy-12H-pyrano[4,3-j]-1,2-benzodioxepin.

WO92/02217 discloses a synergistic antimalarial composition which comprises lumefantrine (benflumetol) and artemether. The compositions disclosed therein include dosage forms such as tablet containing 20 mg artemether and 120 mg lumefantrine.

Artemether and lumefantrine as a fixed dose antimalarial combination is commercially available in the form of a tablet containing 20 mg artemether and 120 mg lumefantrine (20+120 mg tablet). It is sold under the name of Coartem®/Riamet® by Novartis. The dosing regimen for Coartem® is quite complicated and the number of tablets per dose depends on the body weight. In semi-immune patients, the manufacturer recommends the 4-dose regimen, consisting of 1, 2, 3 or 4 tablets taken at 0 h, 8 h, 24 h and 48 h. The total course for an adult is 16 tablets, which gives a total dose of 320 mg of artemether plus 1920 mg of lumefantrine.

In areas with multidrug-resistant P. falciparum and in non-immune patients, an intensive 6-dose course consisting of the doses shown above at 0 h and 8 h, and twice daily doses on the next 2 days is recommended, as shown in Table 1.

Thus, the course for an adult would be 4 tablets at 0 h and 8 h and 4 tablets twice a day on the second and third days.

TABLE 1
Dosage schedule for Artemether and Lumefantrine
Treatment with Coartem ®

| | Number of Tablets per dose | |
|---|---|---|
| Weight | (at 0 h, 8 h, 24 h, | Content Of Artemether (A) and |
| (Kg) | 36 h, 48 h and 60 h) | Lumefantrine (L) per dose |
| <10 | Not recommended | |
| 10-14 | 1 | 20 mg A + 120 mg L |
| 15-24 | 2 | 40 mg A + 240 mg L |
| 25-34 | 3 | 60 mg A + 360 mg L |
| >35 | 4 | 80 mg A + 480 mg L |

Thus, as evident from the table above, the tablet burden remains significant over the course of a day. Further, such a dosing regimen may lead to confusion, low reliability and patient non-compliance.

Therefore, there is a need for a high dose oral pharmaceutical composition, e.g., a tablet, of artemether and lumefantrine having satisfactory dissolution and bioavailability, and which may be provided as a substitute for the multiple tablet intakes per dose and hence improve patient compliance.

Hence, the present invention relates to high dose oral pharmaceutical compositions of artemether and lumefantrine comprising artemether in an amount of from about 40 mg to about 80 mg, lumefantrine in an amount of from about 240 mg to about 480 mg. The compositions provide improved patient compliance and acceptability when compared to the 20+120 mg marketed formulation.

Further, the invention provides divisible tablets of artemether and lumefantrine, having one or more score lines for sectioning of the tablets so that the active substance contained in each tablet can be administered fully or to the extent of half or a quarter, depending on the prescription or requirement.

### SUMMARY OF THE INVENTION

According to one embodiment there is provided a high dose oral pharmaceutical composition of artemether and lumefantrine comprising artemether in an amount of from 40 mg to 80 mg, lumefantrine in an amount of from 240 mg to 480 mg, and one or more pharmaceutically acceptable excipients.

According to another embodiment there is provided a high dose oral pharmaceutical composition of artemether and lumefantrine comprising artemether in an amount of 40 mg, lumefantrine in an amount of 240 mg, and one or more pharmaceutically acceptable excipients.

According to another embodiment there is provided a high dose oral pharmaceutical composition of artemether and lumefantrine comprising artemether in an amount of 60 mg, lumefantrine in an amount of 360 mg, and one or more pharmaceutically acceptable excipients.

According to further embodiment there is provided a high dose oral pharmaceutical composition of artemether and lumefantrine comprising artemether in an amount of 80 mg, lumefantrine in an amount of 480 mg, and one or more pharmaceutically acceptable excipients.

According to another embodiment there is provided a divisible tablet of artemether and lumefantrine comprising artemether in an amount of from 40 mg to 80 mg, lumefantrine in an amount of from 240 mg to 480 mg, and one or more pharmaceutically acceptable excipients, wherein the tablet includes one or more score lines disposed on its surface, which permits the breakage of the tablet into multi sections for consumption.

According to further embodiment there is provided a divisible tablet of artemether and lumefantrine comprising artemether in an amount of 80 mg, lumefantrine in an amount of 480 mg, and one or more pharmaceutically acceptable excipients, wherein the tablet includes two score lines disposed on its surface, which permits the breakage of the tablet into equal quarter sections for consumption.

According to still another embodiment there is provided a method for the treatment of malaria, the method comprising administering to the patient in need thereof a high dose oral pharmaceutical composition of artemether and lumefantrine comprising artemether in an amount of from 40 mg to 80 mg, lumefantrine in an amount of from 240 mg to 480 mg, and one or more pharmaceutically acceptable excipients.

### DETAILED DESCRIPTION OF THE INVENTION

The high dose oral pharmaceutical composition of artemether and lumefantrine comprises artemether in an amount of from 40 mg to 80 mg, lumefantrine in an amount of from 240 mg to 480 mg, and one or more pharmaceutically acceptable excipients. Preferably, the high dose oral pharmaceutical composition comprises artemether in an amount of 40 mg and lumefantrine in an amount of 240 mg; artemether in an amount of 60 mg and lumefantrine in an amount of 360 mg; or artemether in an amount of 80 mg and lumefantrine in an amount of 480 mg.

The term “artemether” as used herein and in the appended claims refers to artemether free base.

The term “lumefantrine” as used herein and in the appended claims refers to lumefantrine or its enantiomers thereof.

The term “divisible tablet” as used herein and in the appended claims refers to tablet having one or more score lines disposed on the surface of the tablet, which permits the breakage of the tablet into multisections for consumption.

The oral pharmaceutical composition may be in the form of tablet or capsule. Preferably the composition is a tablet.

The “pharmaceutically acceptable excipients” may be selected from one or more of binders, diluents, disintegrants, lubricants/glidants, solubilizers/wetting agents and such like.

Suitable binders include polymeric substances having sufficient elasticity and structural stability as a film. Generally the binders may be selected from one or more of cellulose derivatives such as hydroxypropylmethyl cellulose, hydroxypropyl cellulose and methylcellulose; gums such as xanthan gum, gum acacia and tragacanth; water-soluble vinylpyrrolidone polymers such as polyvinylpyrrolidone and copolymer of vinylpyrrolidone vinyl acetate and sugars such as sorbitol and mannitol.

Suitable diluents may be selected from one or more of sugars such as dextrose, glucose and lactose; sugar alcohols such as sorbitol, xylitol and mannitol; cellulose derivatives such as powdered cellulose and microcrystalline cellulose and starches such as corn starch, pregelatinized starch and maize starch.

Suitable disintegrants may be selected from one or more of sodium starch glycolate, croscarmellose sodium, crospovidone and corn starch.

The lubricant/glidants may be selected from one or more of magnesium stearate, talc, sodium stearyl fumarate and colloidal silicon dioxide.

The solubilizers/wetting agents may be selected from one or more of sodium lauryl sulphate and polysorbate 80.

The compositions in the form of tablets or capsule may be prepared by conventional processes known to a person skilled in the art such as by wet granulation, dry granulation or direct compression.

The present invention provides several processes for the preparation of a high dose oral pharmaceutical composition of artemether and lumefantrine.

One process comprises blending artemether and lumefantrine with one or more pharmaceutically acceptable excipients to obtain a blend followed by directly compressing the blend to obtain tablets.

Another process comprises the steps of: a) blending artemether and lumefantrine with one or more pharmaceutically acceptable excipients to obtain a first blend, b) adsorbing one or more wetting agents on one or more pharmaceutically acceptable excipients to obtain a second blend, and, c) mixing the blends obtained in step (a) and step (b) followed by compression to obtain tablets.

A third process comprises the steps of:
a) adsorbing one or more wetting agents on one or more pharmaceutically acceptable excipients to obtain a blend,
b) mixing artemether and lumefantrine with the blend of step 1, followed by compaction to obtain slugs,
c) desizing the slugs obtained in step (b) followed by mixing with one or more pharmaceutically acceptable excipients to obtain a final blend, and,
d) compressing the final blend of step (c) to obtain tablets.

A fourth process comprises the steps of:
a) adsorbing one or more wetting agents on one or more pharmaceutically acceptable excipients to obtain a blend,
b) mixing artemether, lumefantrine and optionally one or more pharmaceutically acceptable excipients, followed by compaction to obtain slugs,
c) desizing the slugs obtained in step (b) followed by mixing with the blend obtained in step (a) and one or more pharmaceutically acceptable excipients to obtain a final blend, and,
d) compressing the final blend of step (c) to obtain tablets.

The high dose oral pharmaceutical compositions of the present invention may be in the form of divisible tablets having one or more score lines disposed on the surface of the tablets. The score lines can be positioned variously about the tablet such as along the top and bottom surfaces thereof. Special placement of score lines permits an accurate equal bisectional, trisectional and quarter sectional fracture of the tablet for patient consumption. Each amount of active substance separated in this manner from the tablet, then constitutes in itself, a new separate dosage of a medicament and is thus governed by the same prescriptions regarding accuracy and permissible limits of dosage, as in the case of the undivided tablet, for e.g., a tablet with a circular horizontal cross-section, having two score lines disposed on the surface, wherein the score lines are placed diametrically and perpendicular to each other, such that the tablet may be fractured into equal quarter sections for consumption.

The present invention also provides high dose oral pharmaceutical composition of artemether and lumefantrine of the present invention, comprising artemether in an amount of 40 mg and lumefantrine in an amount of 240 mg, which may be bioequivalent to two tablets of Coartem®, marketed by Novartis.

The present invention also provides high dose oral pharmaceutical composition of artemether and lumefantrine of the present invention, comprising artemether in an amount of 60 mg and lumefantrine in an amount of 360 mg, which may be bioequivalent to three tablets of Coartem®, marketed by Novartis.

The present invention also provides high dose oral pharmaceutical composition of artemether and lumefantrine of the present invention, comprising artemether in an amount of 80 mg and lumefantrine in an amount of 480 mg, which may be bioequivalent to four tablets of Coartem®, marketed by Novartis.

While the present invention has been described in terms of its specific embodiments, certain modifications and equivalents will be apparent to those skilled in the art and are included within the scope of the present invention.

### Example 1

| | Quantity |
|---|---|
| | (mg/tablet) |
| Ingredients | (80 + 480 mg) |
| Artemether | 80.0 |
| Lumefantrine | 480.0 |
| Microcrystalline cellulose (MCC 112) | 250.0 |
| Hydroxypropyl methylcellulose | 20.0 |
| Croscarmellose sodium | 120.0 |
| Colloidal silicon dioxide | 20.0 |
| Magnesium stearate | 30.0 |
| Tablet weight | 1000.0 |

1. Artemether & Lumefantrine were sifted through specified sieve along with microcrystalline cellulose, hydroxypropyl methylcellulose and croscarmellose sodium and blended in double cone blender to obtain a blend.
2. Blend of a step 1 was further blended with sifted colloidal silicon dioxide and magnesium stearate to obtain a final blend.
3. The final blend of step 2 was compressed using approved tooling to obtain tablets.

### Examples 2-4

| | Quantity | Quantity | Quantity |
|---|---|---|---|
| | (mg/tablet) | (mg/tablet) | (mg/tablet) |
| | Example 2 | Example 3 | Example 4 |
| Ingredients | 40 + 240 mg | 60 + 360 mg | 80 + 480 mg |
| Intragranular | | | |
| Artemether | 40.0 | 60.0 | 80.0 |
| Lumefantrine | 240.0 | 360.0 | 480.0 |
| Microcrystalline cellulose | 42.6 | 63.9 | 85.2 |
| Croscarmellose Sodium | 56.0 | 84.0 | 112.0 |
| Hydroxypropyl | 8.0 | 12.0 | 16.0 |
| methylcellulose | | | |
| Colloidal Silicon dioxide | 10.0 | 15.0 | 20.0 |
| Extragranular | | | |
| Microcrystalline cellulose | 20.0 | 30.0 | 40.0 |
| Polysorbate 80 | 1.0 | 1.5 | 2.0 |
| Microcrystalline cellulose | 56.4 | 84.6 | 112.8 |
| Colloidal Silicon dioxide | 6.0 | 9.0 | 12.0 |
| Magnesium Stearate | 20.0 | 30.0 | 40.0 |
| Total | 500.0 | 750.0 | 1000.0 |

1. Artemether & Lumefantrine were sifted through specified sieve and were mixed geometrically to obtain a mixture.
2. To the mixture obtained in step 1 microcrystalline cellulose, hydroxypropylmethylcellulose, croscarmellose sodium and colloidal silicon dioxide were added to obtain a blend.
3. The blend obtained in step 2 was compacted to obtain slugs, which were sized and sifted through specified sieves to obtain granules.
4. Polysorbate 80 was adsorbed on microcrystalline and sifted through specified sieve to obtain a blend.
5. The blend obtained in step 4 was mixed with the granules obtained in step 3 to obtain a mixture.
6. To the mixture obtained in step 5, sifted microcrystalline cellulose, colloidal silicon dioxide and magnesium stearate were added and mixed to obtain a final blend.
7. The final blend of step 6 was compressed using approved tooling to obtain tablets.
Stability data & Dissolution Profile of the above example is given below:

TABLE 1
Stability Data For Example 4 after 2 months in terms
of assay value showed the following trend:

Example 4: Assay (% w/w)

| | Initial | 2M (40° C./75% RH) |
|---|---|---|
| Artemether | 102.86 | 99.87 |
| Lumefantrine | 103.71 | 102.46 |

The above data shows that artemether and lumefantrine are stable in the composition, soon after preparation. Further, the composition showed acceptable potency even after storage at accelerated condition for about two months.

TABLE 2
Dissolution profile for Example 4

| | |
|---|---|
| | Percentage drug released |
| Time | Example 4 |

| (in min) | Artemether | Lumefantrine |
|---|---|---|
| 15 min. | 97 | 103 |
| 30 min | 97 | 104 |
| 45 min. | 99 | 103 |
| 60 min. | 101 | 104 |

For Artemether: Dissolution profile in 1800 ml pH 7.0 phosphate buffer + 3.0% sodium lauryl sulphate, at 100 rpm using USP type II method.
For lumefantrine: Dissolution profile in 1800 ml 0.1N HCl + 3.0% benzalkonium chloride, at 75 rpm using USP type II method

TABLE 3
Assay value for example 2

| | Example 2 |
|---|---|
| | Assay (% w/w) |
| Artemether | 103.93 |
| Lumefantrine | 100.07 |

TABLE 4
Dissolution profile for Example 2

| | |
|---|---|
| | Percentage drug released |
| Time | Example 2 |

| (in min) | Artemether | Lumefantrine |
|---|---|---|
| 15 min. | 97 | 101 |
| 30 min | 98 | 102 |
| 45 min. | 100 | 103 |
| 60 min. | 101 | 102 |

For Artemether: Dissolution profile in 1800 ml pH 7.0 phosphate buffer + 3.0% sodium lauryl sulphate, at 100 rpm using USP type II method.
For lumefantrine: Dissolution profile in 1800 ml 0.1N HCl + 3.0% benzalkonium chloride, at 75 rpm using USP type II method

While particular high dose oral pharmaceutical composition has been described above, it will be apparent that various modifications and combinations of the composition detailed in the text can be made without departing from the spirit and scope of the invention. For example, additional exemplary tablet formulations are contemplated to make high dose oral pharmaceutical composition of artemether and lumefantrine, as disclosed in the examples 5-12.

### Examples 5-6

| | Quantity | Quantity |
|---|---|---|
| | (mg/tablet) | (mg/tablet) |
| | Example 5 | Example 6 |
| Ingredients | (40 + 240 mg) | (60 + 360 mg) |
| Artemether | 40 | 60.00 |
| Lumefantrine | 240 | 360.0 |
| Microcrystalline cellulose (MCC 112) | 125.00 | 187.5 |
| Hydroxypropyl methylcellulose | 10.00 | 15.00 |
| Croscarmellose sodium | 60.00 | 90.00 |
| Colloidal silicon dioxide | 10.00 | 15.00 |
| Magnesium stearate | 15.00 | 22.5 |
| Tablet weight | 500.0 | 750.0 |

### Procedure:

1. Sift Artemether & Lumefantrine through specified sieve along with microcrystalline cellulose, hydroxypropyl methylcellulose and croscarmellose sodium and blend in double cone blender to obtain a blend.
2. Sift blend of a step 1 with colloidal silicon dioxide and magnesium stearate to obtain a final blend.
3. Compress the final blend of step 2 using approved tooling to obtain tablets.

### Example 7-9

| | Quantity | Quantity | Quantity |
|---|---|---|---|
| | (mg/tablet) | (mg/tablet) | (mg/tablet) |
| | Example 7 | Example 8 | Example 9 |
| Ingredients | (40 + 240 mg) | (60 + 360 mg) | (80 + 480 mg) |
| Artemether | 40.0 | 60.0 | 80.0 |
| Lumefantrine | 240.0 | 360.0 | 480.0 |
| Microcrystalline | 123.76 | 185.64 | 247.52 |
| cellulose (MCC 112) | | | |
| Hydroxypropyl | 10.0 | 15.0 | 20.0 |
| methylcellulose | | | |
| Croscarmellose | 60.0 | 90.0 | 120.0 |
| sodium | | | |
| Polysorbate 80 | 1.24 | 1.86 | 2.48 |
| Colloidal silicon | 10.0 | 15.0 | 20.0 |
| dioxide | | | |
| Magnesium stearate | 15.0 | 22.5 | 30.0 |
| Tablet weight | 500.0 | 750.0 | 1000.0 |

### Procedure:

1. Mix microcrystalline cellulose, hydroxypropyl methylcellulose, and croscarmellose sodium to obtain a mixture.
2. Adsorb polysorbate 80 on to the mixture of step 1, and sift through specified sieve to obtain a blend.
3. Sift Artemether & Lumefantrine through specified sieve and mix with blend of step 2 followed by blending with sifted colloidal silicon dioxide and magnesium stearate to obtain a final blend.
4. Compress the final blend obtained in step 3 using approved tooling to obtain tablets.

### Examples 10-12

| | Quantity | Quantity | Quantity |
|---|---|---|---|
| | (mg/tablet) | (mg/tablet) | (mg/tablet) |
| | Example 10 | Example 11 | Example 12 |
| Ingredients | (40 + 240 mg) | (60 + 360 mg) | (80 + 480 mg) |
| Intragranular | | | |
| Artemether | 40.0 | 60.0 | 80.0 |
| Lumefantrine | 240.0 | 360.0 | 480.0 |
| Microcrystalline | 83.76 | 125.64 | 167.52 |
| cellulose (MCC 112) | | | |
| Hydroxypropyl | 10.0 | 15.0 | 20.0 |
| methylcellulose | | | |
| Croscarmellose | 30.0 | 45.0 | 60.0 |
| sodium | | | |
| Polysorbate 80 | 1.24 | 1.86 | 2.48 |
| Extragranular | | | |
| Croscarmellose | 30.00 | 45.00 | 60.00 |
| sodium | | | |
| Microcrystalline | 40.0 | 60.0 | 80.0 |
| cellulose (MCC 112) | | | |
| Colloidal silicon | 10.0 | 15.0 | 20.0 |
| dioxide | | | |
| Magnesium stearate | 15.0 | 22.5 | 30.0 |
| Tablet weight | 500.0 | 750.0 | 1000.0 |

### Procedure:

1. Mix microcrystalline cellulose, hydroxypropylmethylcellulose, and croscarmellose sodium to obtain a mixture.
2. Adsorb polysorbate 80 on mixture of step 1, and sift through specified sieve to obtain a blend.
3. Sift Artemether & Lumefantrine through specified sieve and mix with the blend of step 2 followed by compaction to obtain the slugs.
4. Deslug the slugs of step 3 followed by desizing and then blended with sifted croscarmellose sodium, microcrystalline cellulose and colloidal silicon dioxide to obtain a mixture.
5. Sift magnesium stearate through specified sieve and blend with the mixture of step 4 to obtain a final blend.
6. Compress the final blend of step 5 using approved tooling to obtain tablets.

We claim:

1. A high dose oral pharmaceutical composition of artemether and lumefantrine comprising artemether in an amount of from 40 mg to 80 mg, lumefantrine in an amount of from 240 mg to 480 mg, and one or more pharmaceutically acceptable excipients.

2. The high dose pharmaceutical composition according to claim 1 comprising artemether in an amount of 40 mg and lumefantrine in an amount of 240 mg.

3. The high dose pharmaceutical composition according to claim 1 comprising artemether in an amount of 60 mg and lumefantrine in an amount of 360 mg.

4. The high dose pharmaceutical composition according to claim 1 comprising artemether in an amount of 80 mg and lumefantrine in an amount of 480 mg.

5. The high dose pharmaceutical composition according to claim 1, wherein the pharmaceutically acceptable excipient is selected from one or more of binders, diluents, disintegrants, lubricants and wetting agents.

6. The high dose pharmaceutical composition of claim 1, wherein the composition is in the form of tablet.

7. The high dose oral pharmaceutical composition of claim 1 wherein the high dose oral pharmaceutical composition is a divisible tablet comprising artemether in an amount of from 40 mg to 80 mg, lumefantrine in an amount of from 240 mg to 480 mg and one or more pharmaceutically acceptable excipients, and wherein the tablet includes one or more score lines disposed on its surface, which permits the breakage of the tablet into multi sections for consumption.

8. The divisible tablet according to claim 7 comprising artemether in an amount of 80 mg, lumefantrine in an amount of 480 mg and one or more pharmaceutically acceptable excipients, wherein the tablet includes two score lines disposed on its surface, which permits the breakage of the tablet into equal quarter sections for consumption.

9. The divisible tablet according to claim 7, wherein the pharmaceutically acceptable excipient is selected from one or more of binders, diluents, disintegrants, lubricants and wetting agents.

10. A process for the preparation of a high dose oral pharmaceutical composition of artemether and lumefantrine, the process comprising the steps of:
a) adsorbing one or more wetting agents on one or more pharmaceutically acceptable excipients to obtain a second blend,
b) mixing the blend obtained in step (a) with artemether and lumefantrine, either individually or as pre-mixed blend, and,
c) compressing the final blend of step (b) to obtain tablets.

11. (canceled)

12. The process according to any of the claims 10, wherein the wetting agent is selected from one or more of sodium lauryl sulphate and polysorbate 80.

13. The process according to any of the claims 10, wherein the pharmaceutically acceptable excipient is selected from one or more of binders, diluents, disintegrants, lubricants and wetting agents.

14. The use of the pharmaceutical composition of preceding claim 1 for the treatment of malaria.

15. The process according to claim 10, wherein the blend obtained in step (b) is compacted to obtain slugs and the slugs are desized to obtain granules.

16. The use of the pharmaceutical composition of claim 7 for the treatment of malaria.
